(19) United States
(12) Patent Application Publication
(10) Pub. No.: US 2008/0027023 A1
(43) Pub. Date: Jan. 31, 2008

(54) Antiproliferative Compositions Comprising Aryl Substituted Xylopyranoside Derivatives

(76) Inventors: Ulf Ellervik, Lund (SE); Lars-Ake Fransson, Lund (SE); Mattias Nils Henry Belting, Lund (SE); Katrin Mani, Lund (SE)

Correspondence Address: YOUNG & THOMPSON, 745 SOUTH 23RD STREET, 2ND FLOOR, ARLINGTON, VA, 22202, US

(21) Appl. No.: 11/632,273

(22) PCT Filed: Jul. 14, 2005
(86) PCT No.: PCT/SE05/01152

§ 371 Date: Mar. 12, 2007

(30) Foreign Application Priority Data
Jul. 15, 2004 (SE) 0401871-9

(51) Int. Cl. A61K 31/715; A61P 35/00; C13K 13/00

(52) U.S. Cl. 514/53; 536/123.13

(57) ABSTRACT

Novel xylose based glycoside compounds that have xylose linked O-, S- or C-glycosidically to an aglycone containing several aromatic rings, and compositions that comprise the novel xylose based glycosides and non-xylose-based anti-tumor agents, pharmaceuticals or dietary supplements. The compounds or compositions are administered to treat proliferative diseases, including various forms of cancer.

Novel compounds (xylose-based glycosides) which alone and in combination with other substances specifically inhibit growth of cancer cells.

### FIELD OF THE INVENTION

The present invention relates to combinations of xylose-containing glycosides with other pharmaceutically active compounds and dietary supplements, to pharmaceutical compositions comprising said combinations, as well as to the use of these combinations for the manufacture of a medicament for the treatment of cancer and proliferative disorders. In another aspect, the present invention relates to novel xylose-containing glycosides, to pharmaceutical compositions comprising said glycosides, and to the use of these glycosides for the manufacture of a medicament for the treatment of cancer and proliferative disorders.

### BACKGROUND OF THE INVENTION

Although improved treatment of certain forms of cancer is now available, for many types of cancer, e.g. malignant gliomas, small cell lung cancer, tumors in the small intestine and metastatic malignant melanoma, no effective curative treatment is available. Accordingly, a very large number of approaches have been tested. One of them is the continuous development of antiproliferative drugs. These include agents targeting DNA and DNA replication, such as alkylating agents, e.g. Alkeran®, DNA-adduct formation, e.g. cisplatin, antimetabolites, e.g. methotrexate, and pyrimidine analogues, e.g. fluorouracil. The development of resistance is a serious drawback for all DNA-directed drugs. Mitosis inhibitors, e.g. vincristine and taxol have also been tested. The therapeutic interval is small in the latter cases, because the drugs target mechanisms that are common to normal as well as tumor cells. Other agents inhibit polyamine synthesis, e.g. α-difluoromethylornithine (DFMO) or growth factor-receptor interaction and heparanase activity, e.g. suramin. Both have had limited success because tumor cells can elicit compensatory mechanisms. A further example is β-D-xylosides having an estrogen aglycon, as disclosed in U.S. Pat. No. 5,104,856, the entire teachings of which are enclosed herein by reference.

WO 01/54702 describes the use of certain xylose containing compounds and at least one anti-tumor agent for synergistic antiproliferative activity. The experiments show that a combination of one xylose containing compound, one polyamine synthesis inhibitor (DFMO) and on anti-tumor agent (suramin) has a synergistic antiproliferative effect on transformed endothelial cells (ECV cells). It should be stressed that the effect is cytostatic, i.e. tumour growth is arrested but cancer cells will not be eliminated.

In the present application it will be shown that when a peracetylated xylose-containing compound is used a cytotoxic effect on cancer cells is obtained, and when at least one xylose containing compound is combined with at least one polyamine synthesis inhibitor and, specifically, the NO-donor spermine-NONOate, a cytotoxic effect on cancer cells is obtained. With certain xyloside containing compounds the toxic effect is selective in that cancer cells are eliminated without adverse effects on normal cells. Other xylosides are preferentially arresting growth of fibroblasts and can be used in the treatment of other proliferative disorders. It will also be shown that peracetylated compounds have a lower ED₅₀ than a non-acetylated counterpart, presumably because acetylated compounds are more readily taken up by cells and deacetylated inside cells, whereby their efflux from the cells is impeded.

It has now surprisingly turned out that when at least one xylose containing compound is combined with at least one polyamine synthesis inhibitor and at least one nitric oxide donating or inducing compound, a toxic effect on cancer cells obtained. The toxic effect is selective in that cancer cells but not normal cells are killed.

### DISCLOSURE OF THE INVENTION

The invention is based on a specific group of xylose-based glycosides all of which are part of the so called carbohydrate-to-protein linkage region which joins sulfated glycosaminoglycans to the core protein of their parent proteoglycans. Such glycosides can serve as primers for glycosaminoglycan synthesis. However, some of them unexpectedly provide a specific synergistic antiproliferative effect on transformed or tumor-derived cells when utilised in combination with specific types of anti-tumor agents, other pharmaceuticals or natural substances or dietary supplements not previously known to have anti-tumor effects, as will be further specified below. The group of xylose-based compounds referred to comprises known as well as novel compounds. The non-xylose-based, anti-tumor agents, pharmaceuticals or dietary supplements referred to are generally known.

More specifically, the present invention is based on a composition comprising non-xylose-based, anti-tumor agents, pharmaceuticals or dietary supplements and a glycoside containing xylose linked O—, S- or C-glycosidically to an aglycone containing several aromatic rings.

Preferably, said aglycon contains at least two carbocyclic structures, of which at least one is aromatic, and where at least two carbocyclic structures are optionally condensed to one carbocyclic structure.

More specifically still, the present invention relates to an antiproliferatively active composition comprising
a) at least one compound having the general formula I: 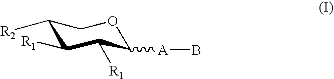
wherein
R₁ groups are same or different and independently selected from N—Y₁Y₂ or O—Y; Y, Y₁ and Y₂ are independently selected from H, alkyl, C(O)aryl, CH₂aryl, C(O)alkyl, C(O)Oalkyl, C(O)Oalkenyl, where the alkyl and alkenyl groups have 1-100 carbon atoms and the aryl group has 6-100 carbon atoms; the C(O)Oalkyl and C(O)Oalkenyl includes preferentially all acyls from acetyl (2 carbon atoms) to heneicosanyl (21 carbon atoms) with or without single or multiple double bonds in all positions.
R₂ is the same as R₁ or R₂ is 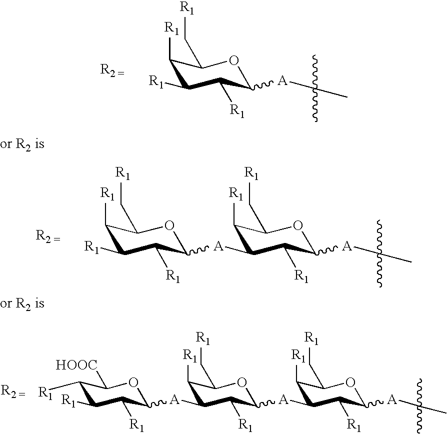
A is O, S, NH or CH₂; B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y; Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms;
and pharmaceutically acceptable salts thereof, in combination with
b) non-xylose compounds chosen from
at least one polyamine synthesis inhibitor;
and at least one nitric oxide donor or stimulator of nitric oxide synthesis or inducer of nitric oxide release from S-nitrosothiols;
and optionally also at least at least one anti-tumor agent selected from the group consisting growth factor-receptor interaction inhibitor or heparanase inhibitor and/or cholesterol traffic inhibitors and/or from the group of inducer of epoxygenase and/or inhibitor of topoisomerase and/or cyanide-donor and/or selene-containing compounds; said combinations of compound(s) a) and agents b) being selected such that a synergistic cytotoxic antiproliferative activity is accomplished. The invention thereby includes the following compounds: 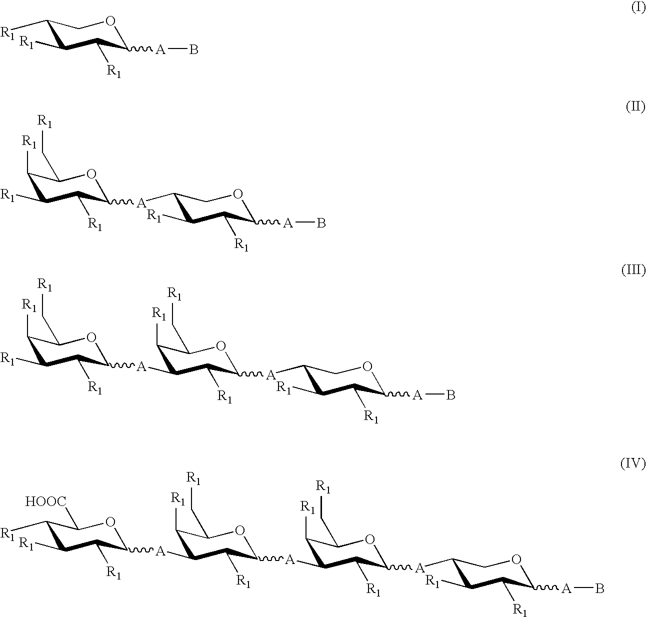
wherein
A is O, S, NH or CH₂; B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y; Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms; R₁ groups are same or different and independently selected from N—Y₁Y₂ or O—Y; Y, Y₁ and Y₂ are independently selected from H, alkyl, C(O)aryl, CH₂aryl, C(O)alkyl, C(O)Oalkyl, C(O)Oalkenyl, where the alkyl and alkenyl groups have 1-100 carbon atoms and the aryl group has 6-100 carbon atoms; the C(O)Oalkyl and C(O)Oalkenyl includes preferentially all acyls from acetyl (2 carbon atoms) to heneicosanoyl (21 carbon atoms) with or without single or multiple double bonds in all positions.

In certain embodiments of the invention, A in compound(s) is O; B is naphthyl substituted with one or more OH-groups, especially selected from n-hydroxynaphthyl, where n is 1, 2, 3, 4, 5, 6, 7 or 8; R₁ are same or different and denote O—Y; Y is independently selected from H or C(O)CH₃.

In some embodiments of the invention, A in compounds is O; B is 6-hydroxy-2-naphthalenyl and preferred compounds are hereinafter referred to as Xyl-2-Nap-6-OH, Gal-4-Xyl-2-Nap-6-OH, Gal-3-Gal-4-Xyl-2-Nap-6-OH and GlcA-3-Gal-3-Gal-4-Xyl-2-Nap-6-OH. R₁ are same or different and denote O—Y; Y is independently selected from H or C(O)CH₃.

In some embodiments of the invention, A in compounds is O; B is 6-hydroxy-2-naphthalenyl and preferred compounds are hereinafter referred to as Xyl-2-Nap-6-OH, Gal-4-Xyl-2-Nap-6-OH, Gal-3-Gal-4-Xyl-2-Nap-6-OH and GlcA-3-Gal-3-Gal-4-Xyl-2-Nap-6-OH.

In a preferred embodiment of the invention R₁ groups in the xylose containing compounds are same or different and independently selected from acyl or OH. Thus, any compound of formula I, II, III or IV above may be partially or fully acylated in this manner. These substances are novel.

The term “peracylated” means that all hydroxyl groups of the corresponding unprotected saccharide have been converted to an ester group. The term “peracetylated” for example therefore means that each available hydroxyl group of the unprotected saccharide has been converted to an acetate group.

By “acyl” in such an ester group is meant —C(O)alkyl or —C(O)alkenyl groups, wherein the alkyl or alkenyl groups have 1-100 carbon atoms especially 1-21 carbon atoms such as an alkyl or alkenyl group having 1, 2, 3, 4, 5, 6, 7, 8, 9, 10, 11, 12, 13, 14, 15, 16, 17, 18, 19, 20 or 21 carbon atoms respectively such as, for example, methyl, ethyl, propyl, butyl, pentyl, hexyl, heptyl, octyl, nonyl, decyl, undecyl, dodecyl, tridecyl, tetradecyl, pentadeyl, hedacecyl, heptadecyl, octadecyl, nonadecyl, eicosanyl heneicosanyl and their isomeric forms.

The alkenyl groups may have a single double bond or multiple double double bonds in 2 or more e.g. in all positions. Example of useful alkenyl groups include linoleyl from linoleic acid (cis,cis-9,12-octadecadienoic acid), linolenyl from linolenic acid (all cis-9,12,15-octadecatrienoic acid), arachidonyl from arachidonic acid (all cis-5,8,11,14-eicosatetraenoic acid) and cis-5,8,11,14,17-eicosapentaenoyl.

The invention also relates to these new substances, compositions comprising these new substances and the use thereof as pharmaceuticals especially for the preparation of a pharmaceutical against proliferative disorders.

Said agents under b) comprise(s) an agent which inhibits synthesis of intracellular polyamines or growth factor-receptor interaction or heparanase or topoisomerase activity or cholesterol traffic or stimulates nitric oxide formation/release or epoxygenase activity or releases cyanide. In the embodiments of the invention, the polyamine synthesis inhibitor can be α-difluoromethylornithine (DFMO), the growth factor-receptor interaction/heparanase inhibitor is preferably suramin and the cholesterol traffic inhibitor is 3β-(2-diethylaminoethoxy)-androstenone (U18666A). In certain embodiments the stimulator of nitric oxide formation is lipopolysaccharide or interferon-γ. A number of suitable nitric oxide donors are known and they are well exemplified in e.g. WHO 96/35416, the teachings of which are incorporated herein by reference.

The nitric oxide donor is preferably selected from nitroglycerin, S-nitrosothiols, isosorbinid, isosorbidmononitrate and compounds of the formula: 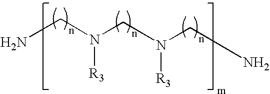
wherein
n may be the same or different and chosen from 0-5,
m is at least 1; preferably between 1 and 10,
R₃ is N(OH)(NO) or H,

Preferably sperminNONOate, wherein m is 1, n is 3, 4 and 3 respectively one R₃ is N(OH)(NO) and the other is H is used.

The inducer of nitric oxide release from S-nitrosothiols is ascorbic acid (vitamin C). The epoxygenase inducer is preferably naphthoflavone, the topoisomerase inhibitor is etoposide, the cyanide donor is amygdalin and the selene-containing compound is selenite.

In one embodiment of the invention the anti-tumor agent(s) b) is (are) selected from suramin and DFMO. Preferably, anti-tumor agent b) is a combination of both suramin and DFMO. Thus, one preferred combination of compound(s) a) and anti-tumor agents b) according to the invention is peracetylated Gal-4-Xyl-2-Nap-6-OH or Xyl-2-Nap-6-OH together with one of suramin and DFMO, or with a combination of both suramin and DFMO.

In another embodiment of the invention the anti-tumor and non-xylose compound b) is(are) selected from DFMO and U18666A. Thus, one preferred combination of compound(s) a) and agents b) according to the invention is peracetylated Gal-4-Xyl-2-Nap-6-OH or Xyl-2-Nap-6-OH together with one of DFMO or U18666A, or with a combination of both DFMO and U18666A.

In another embodiment of the invention the anti-tumour and non-xylose compound b) is(are) selected from DFMO and nitric oxide-donor spermineNONOate, preferably a combination of both. Thus, one preferred combination of compound(s) a) and anti-tumour agent and non-xylose compound b) according to the invention is peracetylated Gal-4-Xyl-2-Nap-6-OH or Xyl-2-Nap-6-OH together with one of DFMO and spermineNONOate, or with a combination of both DFMO and spermineNONOate. The specific synergistic effect of the latter triple combination is a result of 1) DFMO-inhibition of endogenous spermine synthesis resulting in 2) increased spermineNONOate uptake, 3) spontaneous NO-release from spermineNONOate and 4) subsequent potentiation of the antitumour effect of the xylose-containing compound, since these compounds generate products that require NO-catalyzed degradation to achieve maximal antiproliferative effect i.e. even cytotoxic effect. Such products produced synthetically ex vivo cannot be taken up by cells and it is thus an advantage of the present invention that the active products are formed inside cells after uptake of the xylose-containing compounds.

In yet another embodiment of the invention the non-xylose compounds b) is(are) selected from lipopolysaccharide, interferon-γ and ascorbate, preferably a combination of all three. Thus, one preferred combination of compound(s) a) and non-xylose compounds b) according to the invention is peracetylated Gal-4-Xyl-2-Nap-6-OH or Xyl-2-Nap-6-OH together with one of lipopolysaccharide, interferon-γ or ascorbate or, preferably, with a combination of lipopolysaccharide and ascorbate, or lipopolysaccharide, interferon-γ and ascorbate.

In yet another embodiment of the invention the non-xylose compounds b) is(are) selected from DFMO, 30-(2-diethylaminoethoxy)-androstenone and ascorbate, preferably a combination of all three. Thus, one preferred combination of compound(s) a) and non-xylose compounds b) according to the invention is peracetylated Gal-4-Xyl-2-Nap-6-OH or Xyl-2-Nap-6-OH together with one of DFMO, 3β-(2-diethylaminoethoxy)-androstenone or ascorbate or, preferably, with a combination of DFMO, 3β-(2-diethylaminoethoxy)-androstenone and ascorbate.

Furthermore, the present invention relates to the combination of compounds a) and anti-tumor and non-xylose compounds b) as set forth above for use as a pharmaceutical.

Accordingly, the present invention also relates to a pharmaceutical composition comprising a combination of compounds a) and anti-tumor and non-xylose compounds b) as set forth above as active ingredients in association with a pharmaceutically acceptable adjuvant, diluent or carrier.

The combination of compounds (a) and (b) alone or together with other compounds can be administrated by means of oral, subcutaneous or intramuscular delivery, peripheral or central intravenous delivery, intra-arterial, intraventricular, intraperitonial or intrapleural delivery.

In addition, the present invention relates to the use of a combination of compounds a) and anti-tumor and non-xylose compounds b) as set forth above for the manufacture of a medicament for treatment of cancer and proliferative disorders.

The term “proliferative disorders” refers to cell proliferative diseases or conditions and includes any condition characterized by aberrant cell growth, preferably abnormally increased cellular proliferation. Examples of such cell proliferative diseases or conditions include, but are not limited to, cancer, restenosis, and psoriasis inflammation and/or arthritis, atopic dermatitis, asthma, adult respiratory disease, inflammatory bowel disease, Crohn's disease, ulcerative colitis, septic shock, endotoxic shock, gram negative sepsis, toxic shock syndrome, stroke, cardiac and renal reperfusion injury, glomerulonephritis, thrombosis, Alzheimer's disease, graft vs. host reaction, allograft rejections, malaria, acute respiratory distress syndrome, delayed type hypersensitivity reaction, atherosclerosis, cerebral and cardiac ischemia, osteoarthritis, multiple sclerosis, restinosis, angiogenesis, osteoporosis, gingivitis, respiratory viruses, herpes viruses, hepatitis viruses, HIV (i.e., AIDS), Kaposi's sarcoma associated virus, meningitis, cystic fibrosis, pre-term labor, cough, pruritis, multi-organ dysfunction, trauma, strains, sprains, contusions, psoriatic arthritis, herpes, encephalitis, CNS vasculitis, traumatic brain injury, CNS tumors, subarachnoid hemorrhage, post surgical trauma, interstitial pneumonitis, hypersensitivity, crystal induced arthritis, acute and chronic pancreatitis, acute alcoholic hepatitis, necrotizing enterocolitis, chronic sinusitis, angiogenic ocular disease, ocular inflammation, retinopathy of prematurity, diabetes I and II, diabetic retinopathy, macular degeneration with the wet type preferred and corneal neovascularization, polymyositis, vasculitis, acne, gastric and duodenal ulcers, celiac disease, esophagitis, glossitis, airflow obstruction, airway hyperresponsiveness, bronchiectasis, bronchiolitis, bronchiolitis obliterans, chronic bronchitis, cor pulmonae, cough, dyspnea, emphysema, hypercapnea, hyperinflation, hypoxemia, hyperoxia-induced inflammations, hypoxia, surgical lung volume reduction, fibrotic conditions, such as chronic obstructive lung disease (COLD), also known as chronic obstructive pulmonary disease (COPD), pulmonary fibrosis, pulmonary hypertension, right ventricular hypertrophy, peritonitis associated with continuous ambulatory peritoneal dialysis (CAPD), granulocytic ehrlichiosis, sarcoidosis, small airway disease, ventilation-perfusion mismatching, wheeze, colds, gout, alcoholic liver disease, lupus, burn therapy, periodontitis, transplant reperfusion injury and early transplantation, in wound healing and for a stimulating effect on PDGF (platelet derived growth factor).

The invention especially relates to adenocarcinoma or small cell carcinoma of the lung, adenocarcinoma of the stomach, colon, liver, prostate and breast, malignant melanoma and glioma.

For the treatment of certain of the above diseases one may only whish to stop proliferation but not reach to a cytotoxic effect. This may especially hold true for psoriasis and fibrotic conditions such as COLD, COPD and pulmonary fibrosis. These conditions may be treated with a low dose of the active components of the composition according to the invention. They may also be treated with just one or two of the compounds mentioned under a) and/or b) above.

Thus one compound belonging to group a) may be used together with one or more compounds chosen from a polyamine synthesis inhibitor or with one or more compounds chosen from nitric oxide donors or nitric oxide stimulators.

The present invention is also concerned with a method for the treatment of cancer and proliferative disorders, wherein said method comprises administering of a therapeutically effective amount of a combination of compound(s) a) and anti-tumor and non-xylose compounds b) as set forth above to a human or animal patient.

The typical dosage of said compound(s) a) and anti-tumor and non-xylose compounds b) vary within a wide range and will depend on various factors, such as the particular requirement of each receiving individual and the route of administration. However, the dosages are generally within the range of 0.001-100 mg/kg body weight for a) and b) each.

The concept of synergism of the present invention may be further illustrated by the following Scheme 1:

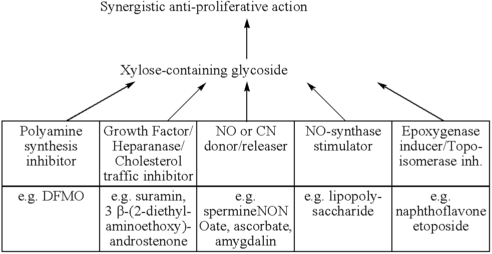

In a different aspect, the present invention also relates to further novel compounds having the general formulas I-IV: 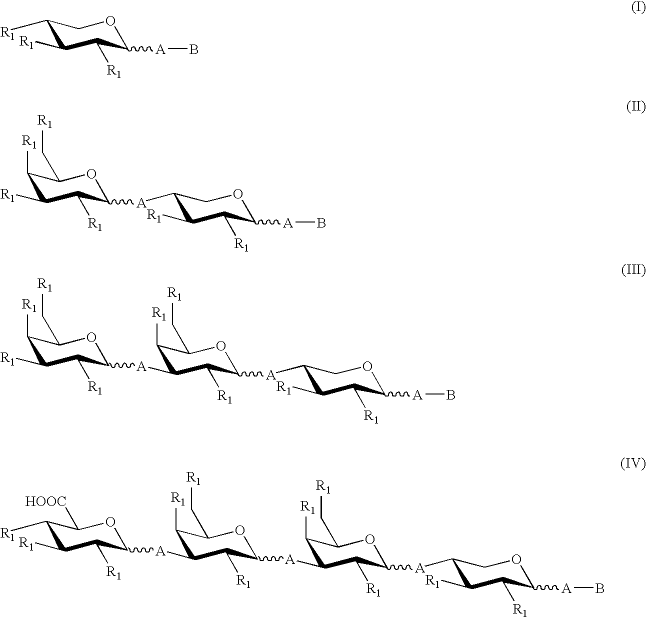
wherein
R₁ groups are same or different and independently selected from N—Y₁Y₂ or O—Y; Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms;
A is O, S, NH or CH₂; B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y; Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms; with the exclusion of compounds with the general formula I and R₁ is OH, A is O and B is naphthyl with 0, 1 or 2 OH-groups; or R₁ is OH, A is S and B is naphthyl.

As to the preferred embodiments of the various groups and substituents of the novel compounds according to the invention, these are the same as those described earlier in regard to compounds (a) in said combinations of compounds (a) and agents (b).

The invention also relates to a composition comprising these new compounds, especially a pharmaceutical composition. Further, the invention relates to the use of these new substances for the preparation of a pharmaceutical for use as an antiproliferative agent. The antiproliferative effect may be against any of the diseases mentioned above.

The compounds (a) and agents (b) described above can also be used in combination with other antiproliferative, alkylating agents, plant alkaloids and natural products like topoisomerase inhibitors, mitiotic inhibitors and enzymes, antimetabolites and targeted therapies like inhibitors of angiogenesis, endocrine agents and other agents like tamoxifen, leuprolide and flutamine.

The compounds as set forth above are, per se, useful as active ingredients in pharmaceutical compositions, for manufacture of medicaments against cancer and proliferative disorders and for methods of treatment of cancer and proliferative disorders, to the same extent as the combinations of compounds (a) and agents (b) described earlier and combinations of existing and/or future medicaments that will be developed for the treatment of such diseases. Cancer forms that can be treated in this manner include malignant gliomas, non-small lung cancer, bladder carcinomas, liver carcinomas and virally-induced cancer. Other proliferative disorders that can be treated include psoriasis as well as fibrosis associated with chronic inflammation such as asthma, chronic obstructive lung disease, atherosclerotic plaque formation and liver chirrhosis.

The compounds (a) and agents (b) described above can also be used in combination with radiation therapy and heat theraphy.

### BRIEF DESCRIPTION OF THE FIGURES AND TABLES

FIG. 1A shows in histogram form the effect of DFMO, spermineNONOate and a combination of DFMO and spermineNONOate on growth of rat C6 glioma cells in culture. FIG. 1 B shows the effect of various concentrations of Xyl-2-Nap-6-OH alone or in combination with DFMO, in combination with spermineNONOate or in combination with both DFMO and spermineNONOate on growth of rat C6 glioma cells.

FIG. 2 shows the effect of various concentrations of Xyl-2-Nap-6-OH alone or in combination with DFMO, in combination with spermineNONOate or in combination with both DFMO and spermineNONOate on growth of human bladder carcinoma T24 cells previously marketed as endothelial ECV 304 cells.

FIG. 3 shows the effect of various concentrations of Xyl-2-Nap-6-OH alone or in combination with DFMO, in combination with spermineNONOate or in combination with both DFMO and spermineNONOate on growth of normal human lung fibroblasts.

FIG. 4 shows the effect of DFMO, spermineNONOate, a combination of DFMO and spermineNONOate and of various concentrations of peracetylated Gal-4-Xyl-2-Nap-6-OH alone or in combination with DFMO and spermineNONOate on growth of rat C6 glioma cells.

FIG. 5 shows the effect of various concentrations of peracetylated Xyl-2-Nap-6-OH on (A) human bladder carcinoma T24 cells or (B) normal human lung fibroblasts.

FIG. 6 shows in a scatter plot form the effect of Xyl-2-Nap-6-OH alone or in combination with DFMO on tumour weight or size after inoculation of rat C6 glioma cells implanted subcutaneously in SCID mice (A and C) or intracerebrally in rats (B). Treatment with xyloside was daily contralateral subcutaneous injections (1.7 mM solution; 0.5 ml for mice, 5 ml for rats) for 2 weeks. DFMO (0.1% w/v) was administered via the drinking water.

FIG. 7 depicts the structures of all the 14 isomeric xylosylated dihydroxynaphthalenes.

Table 1 shows the antiproliferative activity of the 14 isomeric xylosylated dihydroxynaphthalenes.

While the invention has been described in relation to certain disclosed embodiments, the skilled person may foresee other embodiments, variations, or combinations which are not specifically mentioned but are nonetheless within the scope of the appended claims.

All references cited herein are hereby incorporated by reference in their entirety.

The expression “comprising” as used herein should be understood to include, but not be limited to, the stated items.

The invention will now be described by way of the following non-limiting examples.

### EXAMPLES

### Preparation of Novel Xylose-Containing Glycosides

NMR-spectra were recorded with a Bruker DRX-400 (400 MHz) instrument and FAB-MS spectra with a JEOL SX-120 mass spectrometer. Chemical shifts are given in ppm downfield from the signal of SiMe₄, with reference to internal CHD₂OD. Reactions were monitored with TLC glass plates coated with silica gel Merck 60 F₂₅₄ and visualized using either UV-light or a solution of orcinol (400 mg/L) in 10% aqueous H₂SO₄. Flash chromatography was performed using Grace Amicon silica gel (35-70 μm). Solid phase extraction (SPE) was performed using IST Isolute™ SPE column 500 mg PH.

The large-scale preparation of Xyl-2-Nap-6-OH (10 g) used for animal experiments was made according to Mani et al. (Glycobiology, 2004, 14, 387-397). Monosaccharidic derivatives were prepared according to Jacobsson et al. (Tetrahedron Lett., 2002, 43, 6549-6552), Mani et al. (Glycobiology, 2004, 14, 387-397), and PCT application Reg. No. 15463.

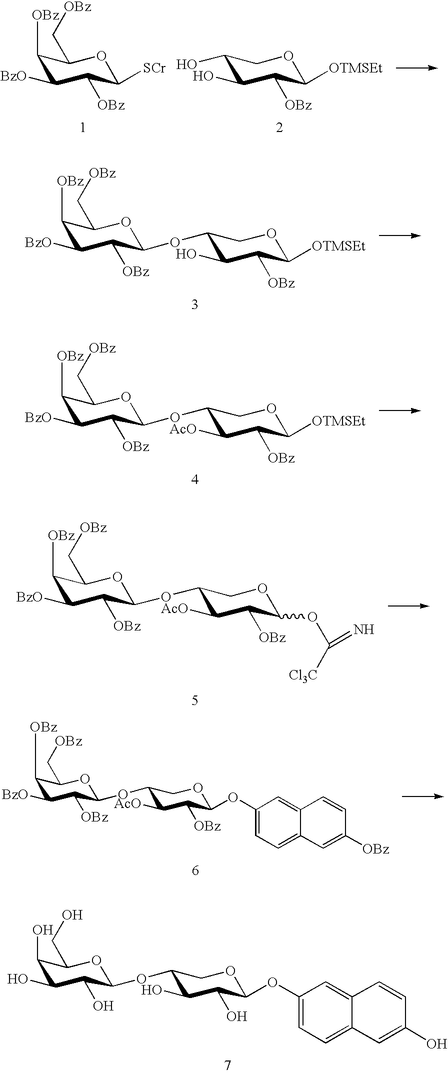

Disaccharidic compounds were prepared according to the following procedure:

### 2-(Trimethylsilyl)ethyl 2-O-benzoyl-4-O-(2,3,4,6-tetra-O-benzoyl-β-D-galactopyranosyl)-β-D-xylopyranoside (3)

A mixture of the thiogalactoside 1 (232 mg, 0.339 mmol), xylopyranoside 2 (100 mg, 0.283 mmol), silver trifluoromethanesulphonate (90.9 mg, 354 mmol) and molecular sieves (325 mg, MS AW 300) were dissolved in dry MeCN (2.5 mL) and CH₂Cl₂ (2.0 mL) and cooled to −78° C. under stirring. A 1.0 M solution of iodine monochloride was added dropwise to the cooled mixture. After 90 min, diisopropylamin (0.60 mL) and the mixture were filtered and flash chromatographed (SiO₂, toluene/EtOAc 10:1) to give 3 (155 mg, 0.166 mmol, 59%).

¹H NMR data (CDCl₃): δ 7.25-8.20 (m, 25H), 6.07 (d, 1H, J=3.4 Hz, H-4′), 5.92 (dd, 1H, J=10.5, 6.0 Hz, H-2′), 5.70 (dd, 1H, J=10.5, 3.4 Hz,

H-3′), 5.25 (dd, 1H, J=9.2, 7.6 Hz, H-2), 5.05 (d, 1H, J=8.0 Hz, H-1′), 4.72 (dd, J=8.0 Hz, H-6′), 4.59 (d, 1H, J=7.6 Hz, H-1), 4.50 (m, 1H, H-6′), 4.24 (d, 1H, J=2.2 Hz, H-4), 4.05 (t, 1H, J=9.1, 2.2 Hz, H-3), 3.83-4.01 (m, 3H, H-5, H-5′, CH₂), 3.55-3.63 (m, 1H, J=13.5, 6.5, 6.4 Hz, CH₂), 3.39-3.47 (m, 1H, J=2.0, H-5), 2.46 (s, 1H, OH), 0.85-1.02 (m, 2H, CH₂), 0 (s, 9H, CH₃).

### 2-(Trimethylsilyl)ethyl 3-O-acetyl-2-O-benzoyl-4-O-(2,3,4,6-tetra-O-benzoyl-β-D-galactopyranosyl)-β-D-xylopyranoside (4)

Compound 3 (28 mg, 0.0300 mmol) was acetylated over night with acetic anhydride (0.7 mL) and pyridine (0.5 mL). The mixture was concentrated and coconcentrated with toluene, and the residue was chromatographed (SiO₂, toluene/EtOAc 5:1) to give 4 (28.9 mg, 99%).

¹H NMR data (CDCl₃): δ 7.40-8.02 (m, 25H), 6.05 (d, 1H, J=2.7 Hz, H-4′), 5.84 (dd, 1H, J=10.4, 7.9 Hz, H-2′), 5.66 (dd, 1H, J=10.4, 3.4 Hz, H-3′), 5.48 (t, 1H, J=8.2 Hz, H-3), 5.21 (dd, 1H, J=8.8, 7.0 Hz, H-2), 5.02 (d, 1H, J=7.9 Hz, H-1′), 4.60 (m, 2H, H-1, H-6′), 4.50 (m, 1H, H-6′), 4.42 (dd, 1H, J=7.1, 6.3 Hz, H-5′), 4.02-4.10 (m, 2H, H-4, H-5), 3.90-3.98 (m, 1H, CH₂), 3.51-3.60 (m, 1H, CH₂), 3.37-3.46 (m, 1H, H-5), 2.00 (s, 3H, AcO), 0.84-1.06 (m, 2H, CH₂), 0 (s, 9H, CH₃).

### 3-O-acetyl-2-O-benzoyl-4-O-(2,3,4,6-tetra-O-benzoyl-β-D-galactopyranosyl)-β-D-xylopyranosyl trichloroacetimidate (5)

Compound 4 (150 mg, 0.154 mmol) was dissolved CH₂Cl₂ (0.9 mL), trifluoroacetic acid (1.7 mL) was added and the mixture was stirred for 90 min. n-propyl acetate (2.0 mL) and 4.0 mL) were added, and the mixture was concentrated. The crude product was dissolved in CH₂Cl₂ (2.7 μL), Cl₃CCN (0.455 mL, 4.5 mmol) and DBU (0.017 mL, 0.113 mol) were added to the solution at 0° C. under Ar. After 90 min, the mixture was concentrated and the residue was chromato-graphed (SiO₂, heptane/EtOAc 2:1) to give 5 (115 mg, 0.113 mmol, 73.4%) as an anomeric mixture (α/β 3:2).

¹H NMR data (CDCl₃): δ 7.20-8.40 (m, 25H), 6.56 (d, 1H, J=3.7 Hz), 6.11 (d, 1H, J=3.7 Hz), 6.02 (d, 1H, J=3.1 Hz), 5.98 (d, 1H, J=3.3 Hz), 5.82 (dd, 1H, J=14.7, 9.5 Hz), 5.81 (dd, 2H, J=19.1, 9.9 Hz), 5.61 (dd, 2H, J=10.4, 3.4 Hz), 5.53 (t, 1H, J=6.0 Hz), 5.36 (dd, 1H, J=5.8, 4.8 Hz), 5.22 (dd, 1H, J=10.2, 3.7 Hz), 5.00 (dd, 2H, J=14.7, 7.9 Hz), 4.67 (dd, 1H, J=10.9, 6.2 Hz), 4.33-4.47 (m, 5H), 4.10-4.25 (m, 2H), 4.00 (m, 1H), 3.89 (dd, 1H, J=11.4, 5.6 Hz), 3.78 (d, 1H, J=11.1), 3.69 (m, 1H), 2.04 (s, 3H), 1.98 (s, 3H)

### 2-(6-benzoyloxynaphthyl)3-O-acetyl-2-O-benzoyl-4-O-(2,3,4,6-tetra-O-benzoyl-β-D-galactopyranosyl)-β-D-xylopyranoside (6)

Boron trifluoride etherate (0.002 mL, 0.016 mmol) was added to a solution of compound 5 (42 mg, 0.0412 mmol) and 6-benzoyloxy-2-hydroxynaphthalene (15.2 mg, 0.0577 mmol) in dry CH₂Cl₂ (0.7 mL) and dry MeCN (1.0 mL) at 0° C. under Ar. After 30 min, Et₃N (0.2 mL) was added and the mixture was concentrated and the residue was chromatographed (SiO₂, heptane/EtOAc; (6:1), (5:1), (4:1), (3:1), (2:1)) to give 6 (42 mg, 0.037 mmol, 90%).

¹H NMR data (CDCl₃): δ 7.15-8.25 (m, 31H, Ar), 6.02 (d, 1H, J=3.4 Hz, H-4′), 5.86 (dd, 1H, J=10.4, 8.0 Hz, H-2′), 5.64 (dd, 1H, J=10.4, 7.0 Hz, H-3′), 5.58 (t, 1H, J=7.2 Hz, H-3), 5.47 (dd, 1H, J=7.4, 5.8 Hz, H-2), 5.42 (d, 1H, J=5.8 Hz, H-1), 5.05 (d, 1H, J=8.0 Hz, H-1), 4.55 (dd, 1H, J=10.4, 6.0, H-6′), 4.46 (d, 1H, J=6.2, H-6′), 4.42 (d, 1H, J=6.8 Hz, H-5′), 4.19 (dd, 1H, J=12.0, 4.5 Hz, H-4), 4.12 (m, 1H, H-5), 3.62 (dd, 1H, J=12.0, 7.4 Hz, H-5), 2.06 (s, 3H, AcO).

### 2-(6-hydroxynaphthyl)-4-O-(β-D-galactopyranosyl)-β-D-xylopyranoside (7)

Methanolic sodium methoxide (1 M, 1.0 mL) was added to a solution of compound 6 in MeOH (1.0 mL). The mixture was stirred at room temperature for 30 min and then neutralized with acetic acid (0.15 mL) and concentrated. The product was purified with reverse phase HPLC to give 7 (8 mg, 0.018 mmol, 85%).

Cellular Growth Assays

Cells were obtained from American type culture collection (ATCC, Rockville, Md.). Regular cell culture media, L-glutamine, penicillin-streptomycin, trypsin, and donor calf serum were obtained from Life Technologies. Dulbeccos Modified Earles Medium, medium 199 and Ham's F-12 medium were purchased from Sigma, difluoromethylornithine (DFMO) was from ILEX Oncology, San Antonio, Tex. Epidermal growth factor was purchased from Genzyme, Cambridge, Mass. and crystal violet from Merck, Germany.

Cells were cultured as monolayers in Dulbeccos Modified Earles Medium (DMEM) supplemented with 10% (v/v) fetal bovine serum, 2 mM L-glutamine, penicillin (100 U/mL) and streptomycin (100 μg/mL) in an incubator with humidified atmosphere and 5% CO₂ at 37° C.

The growth assay procedure has been described elsewhere (Mani et al., 1998, Cancer Res. 58: 1099-1104). Cells were seeded into 96-well microculture plates at 3000 cells/well in DMEM supplemented with insulin (10 ng/mL), transferrin (25 ng/mL) and 10% fetal calf serum. After 4 h of plating the cells were placed in serum-free Ham's F-12 medium supplemented with insulin (10 ng/mL) and transferrin (25 ng/mL) for an additional 24 h. Cells were then allowed to proliferate supported by 10 ng/mL of epidermal growth factor in the presence of 0.01, 0.025, 0.05, 0.1, and 0.2 mM of xyloside. In some experiments cells were pre-treated with DFMO to up-regulate spermine uptake (Belting et al., 2003, J. Biol. Chem. 278: 47181-47189). Controls without growth factor as well as solvent (DMSO) controls were included. The total time of exposure to the various agents was 5 days. Cells were fixed in 1% glutaraldehyde dissolved in Hanks balanced salt solution (NaCl 80 g/L, KCl 4 g/l, glucose 10 g/L, KH₂PO₄ 600 mg/L, NaHPO₄ 475 mg/L) for 15 min, then cell nuclei were stained with 0.1% crystal violet. After washing and cell lysis for 24 h in Triton X-100, the amount of bound dye was measured at A₆₀₀ in a microplate photometer (Titertek multiscan). The results are presented as the difference between A₆₀₀ obtained after 5 days and that obtained immediately after seeding of the cells (t=0). A good correlation between A₆₀₀ and cell number has been demonstrated previously (see Mani et al., 1998).

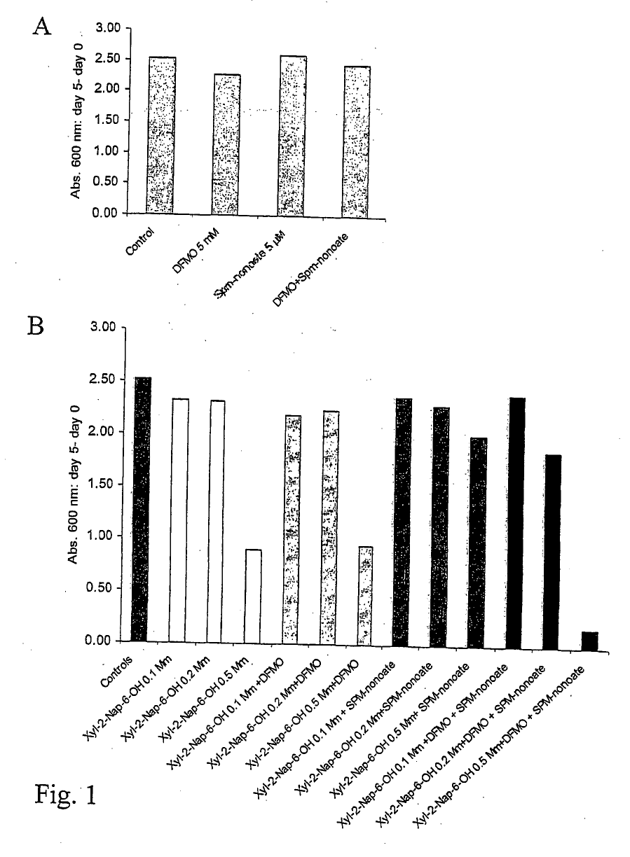

Referring to FIG. 1, the results of treating rat C6 glioma cells with a combination of Xyl-2-Nap-6-OH, DFMO and spermineNONOate are shown. Proliferation of untreated cells is used as reference (control). DFMO inhibits polyamine synthesis and makes cells dependent on uptake of polyamines (spermine, spermidine or putrescine) from the environment. Thus, when cells are treated with DFMO, spermineNONOate is taken up by the cells and releases NO intracellularly with a half-life of 4 h. The results of the experiments with DFMO and spermineNONOate (FIG. 1 A) show that 5 mM DFMO or 5 μM spermineNONOate alone or a combination of 5 nM DFMO and 5 μM spermineNONOate have no significant growth-inhibitory effect compared to untreated cells (control). The results of the experiments described in FIG. 1 B show that 0.5 mM Xyl-2-Nap-6-OH alone inhibits growth by 65%, but a combination of 0.5 mM Xyl-2-Nap-6-OH, 5 mM DFMO and 5 μM spermineNONOate inhibits growth by 93%, a clear synergistic effect.

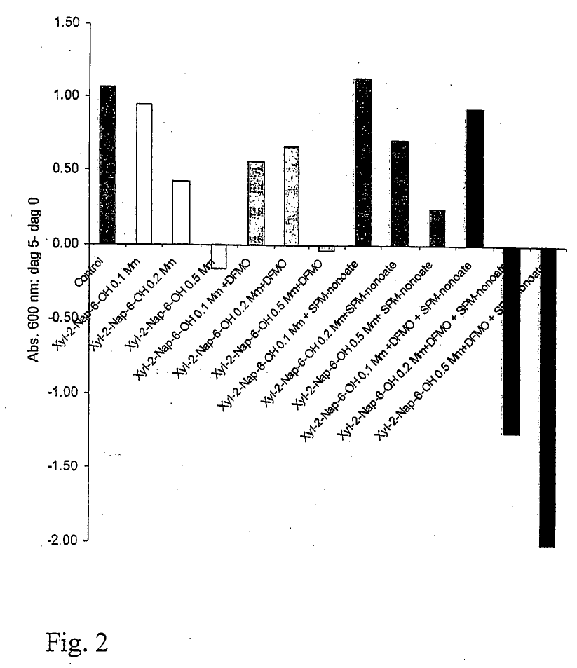

Referring to FIG. 2, results of corresponding experiments with human bladder carcinoma T24 cells are shown. These cells are completely growth-arrested by 0.5 mM Xyl-2-Nap-6-OH but marginally affected by 0.2 mM Xyl-2-Nap-6-OH. However, treatment with a combination of 0.2 mM Xyl-2-Nap-6-OH, 5 mM DFMO and 5 μM spermineNONOate results in complete cell death, i.e. a cytotoxisk effect that has not previously been obtained with these cells (when designated ECV 304). It should be added that a dose of 2.5 5 μM spermineNONOate in combination with 0.2 mM Xyl-2-Nap-6-OH and 5 mM DFMO is insufficient.

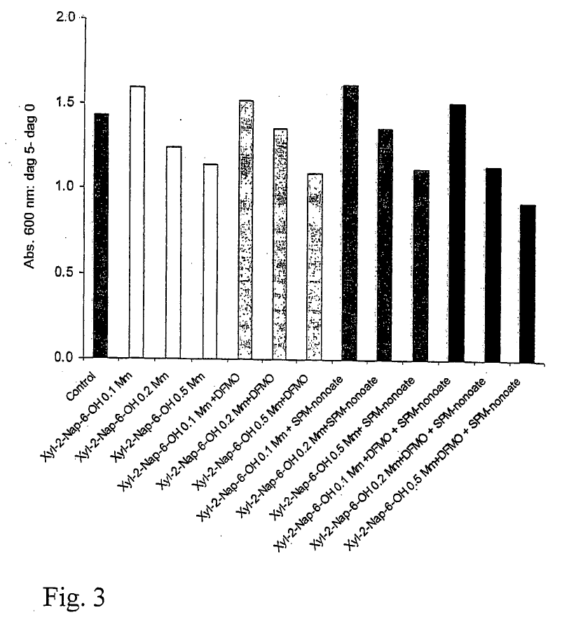

These synergistic effects are not seen in an analogous experiment conducted with normal, untransformed human lung fibroblasts (FIG. 3). Treatment with 0.2 mM Xyl-2-Nap-6-OH, 5 mM DFMO and 5 μM spermineNONOate results only in a 20% reduction of cell growth.

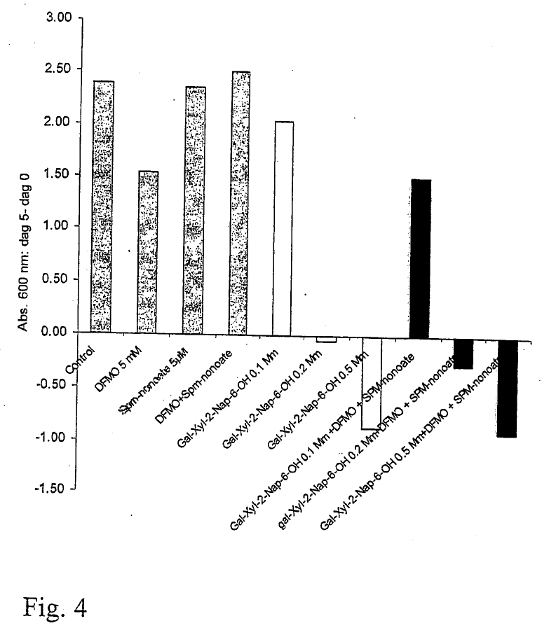

Referring to FIG. 4, the results of treating rat C6 glioma cells with peracetylated Gal-4-Xyl-2-Nap-6-OH are shown. At a concentration of 0.2 mM peracetylated Gal-4-Xyl-2-Nap-6-OH, complete growth-arrest is obtained. In combination with 5 mM DFMO and 5 μM spermineNONOate there is also a significant cytotoxic effect. By comparing these results with the results obtained with Xyl-2-Nap-6-OH (FIG. 1 B), it can be concluded that peracetylated Gal-4-Xyl-2-Nap-6-OH is at least 5-fold more potent than Xyl-2-Nap-6-OH at the same concentrations. The reason for this difference is that peracetylated Gal-4-Xyl-2-Nap-6-OH is deacetylated by non-specific esterases after uptake into cells, whereby the resulting non-acetylated Gal-4-Xyl-2-Nap-6-OH remains trapped inside the cell resulting in a much higher local concentration. Accordingly, non-acetylated Gal-4-Xyl-2-Nap-6-OH is not taken up when added to cells.

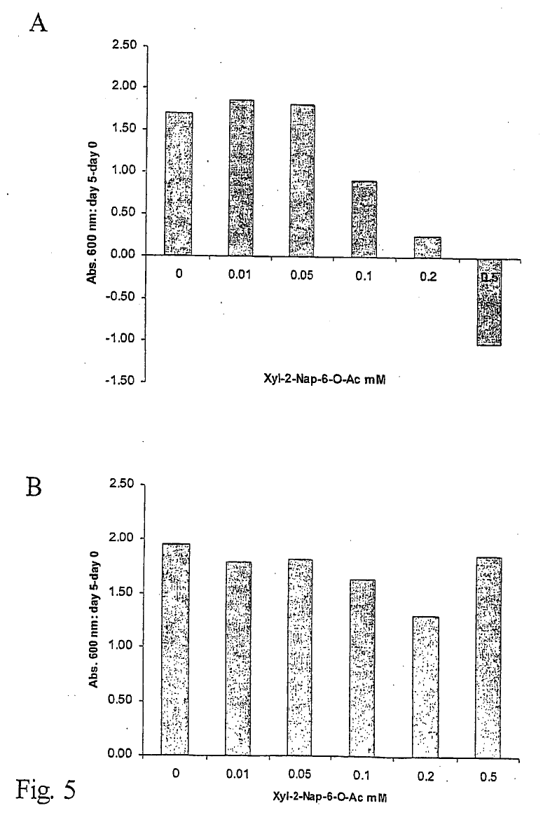

Referring to FIG. 5, the results of treating (A) human bladder carcinoma T24 cells or (B) normal human lung fibroblasts with peracetylated Xyl-2-Nap-6-OH are shown. At a concentration of 0.5 mM peracetylated Xyl-2-Nap-6-OH, a clear cytotoxic effect on T24 cells is obtained (FIG. 5 A) as opposed to non-acetylated Xyl-2-Nap-6-OH which is marginally cytotoxic (FIG. 2). Peracetylated Xyl-2-Nap-6-OH does not affect growth of normal human fibroblasts at the same concentration (FIG. 5 B). Apparently, both non-acetylated and peracetylated Xyl-2-Nap-6-OH attain an equilibrium between the inside and the outside of the cell. However, peracetylated Xyl-2-Nap-6-OH is probably more efficiently taken up than non-acetylated Xyl-2-Nap-6-OH.

It should also be added that Xyl-2-Nap-6-OH selectively inhibits growth of A549 cells, which are currently used as model cells for non-small lung cancer, and SV40 virus infected fibroblasts, and HepG2 hepatoma cells (Mani et al. Glycobiology 14: 387-397). Synergistic cytotoxic effects between xyloside, DFMO and spermine-NONOate have also been observed for these cell lines.

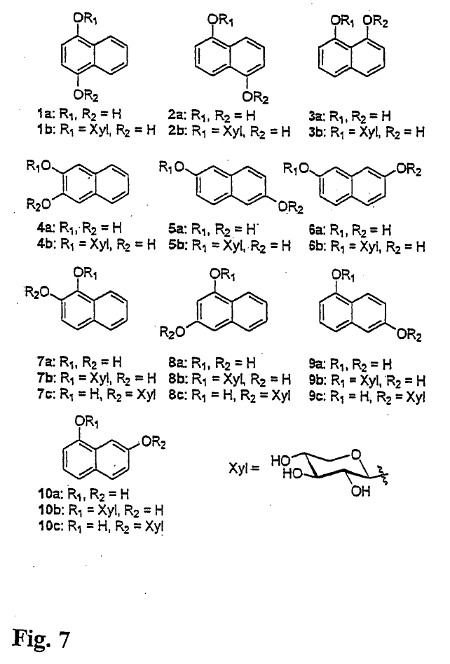

FIG. 7 depicts the structures of all the 14 isomeric xylosylated dihydroxynaphthalenes that have been synthesized and tested. As shown in Table 1, 4 of these (5b, 8b, 8c and 9b) selectively inhibit growth of T24 carcinoma cells. Synergistic cytotoxic effects between these xylosides, DFMO and spermine-NONOate have also been observed. One of these xylosides (4b) has greater effect on fibroblasts than on carcinoma cells and, together with 3b, 7b, and 7c, are potential anti-fibrotic drugs.

Animal Experiments

Female SCIDnodCA mice (7-8 weeks old) or Fisher rats (12 week old) were kept under pathogen-free conditions in the animal barrier facility at the Biomedical Center, Lund University, according to the Swedish guidelines for humane treatment of laboratory animals. The experimental set-up was approved by the ethical committee for animal research in Malmö/Lund, Sweden. To generate tumors, rat C6 glioma cells or human bladder T24 carcinoma cells (1×10⁶ cells in 200 μl PBS) were injected subcutaneously in the dorsal region of 7-8 week old mice (n=4-8) or intracerebrally in rats.

In the present studies, animals (n=8) received 1.7 mM Xyl-2-Nap-6-OH, i.e. a saturated solution, either ad libitum via the drinking water, or by daily injections either subcutaneously (0.5 ml for mice, 5 ml for rats) or intraperitoneally (0.5 ml) for a total period of up to 2 weeks. Controls received drinking water with no additives or daily injections with sterile water. Food and water intake and changes in body weight were monitored. The xyloside was administered either concurrent with or 3 weeks post-injection. In the latter case, the animals were randomly divided into control and treatment groups with no significant differences in tumor volume. The animals were sacrificed following 2-3 weeks of treatment, and tumor mass or volume was recorded.

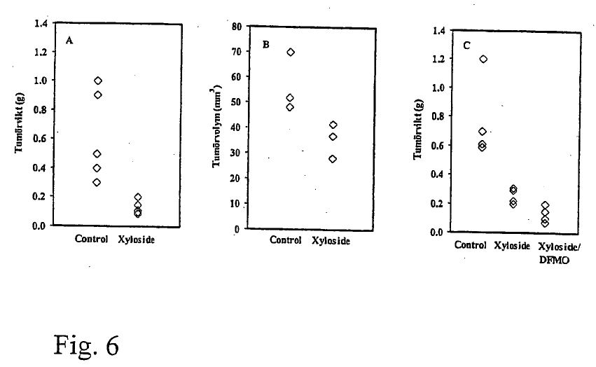

Referring to FIG. 6, the results of treating tumor-bearing mice and rats with Xyl-2-Nap-6-OH are shown. The subcutaneous C6 glioma tumor weight is reduced by 78% in mice by treatment with Xyl-2-Nap-6-OH (FIG. 6 A). Tumor volume is also reduced in the rat model (FIG. 6 B), indicating that Xyl-2-Nap-6-OH can penetrate the blood-brain barrier and reach tumor cells located inside the brain. If tumor-bearing mice treated with Xyl-2-Nap-6-OH also receives DFMO via the drinking-water (0.1% w/v), tumor-load is reduced by 83% (FIG. 6 C). To achieve the same effect with DFMO alone, a 10-20-fold higher concentration of DFMO in the drinking water is required.

Other animal experiments further indicate that Xyl-2-Nap-6-OH, administered in various ways including oral administration, is adsorbed and made available to tumor cells located subcutaneously or orthotopically. Treatment with Xyl-2-Nap-6-OH reduce the average tumor load by 70-97% in mice receiving the compound concomitant with tumor cell inoculation as well as in mice with pre-formed tumors. As virtually all cells in the body have the capacity to take up xylosides, the lack of obvious toxic side-effects in vivo suggests that non-tumor cells are unable to transform these compounds into antiproliferative products in significant amounts. Side-effects via inhibition of proteoglycan synthesis in cartilage and connective tissues is unlikely, as xyloside concentrations greater than 2 mM are required for their inhibition in vitro (Moses et al., 1999, Eur, J. Biochem. 26: 879-884).

TABLE 1
Antiproliferative activity (ED₅₀, mM) of naphthoxylosides towards
HFL-1 cells and T24 cells and HPLC retention times.

| | | | Retention time |
|---|---|---|---|
| Compound | HFL-1^{a} | T24^{a} | (min) |
| 1b | 0.006 | 0.001 | 14.2 |
| 2b | 0.50 | 0.47 | 13.5 |
| 3b | 0.18 | 0.22 | 20.9 |
| 4b | 0.19 | 0.40 | 18.8 |
| 5b | 0.50 | 0.10 | 12.9 |
| 6b | 0.50 | 0.50 | 14.8 |
| 7b | 0.08 | 0.15 | 20.2 |
| 7c | 0.04 | 0.16 | 20.4 |
| 8b | 0.24 | 0.10 | 16.0 |
| 8c | 0.32 | 0.025 | 17.7 |
| 9b | 0.37 | 0.125 | 14.8 |
| 9c | 0.50 | 0.60 | 13.7 |
| 10b | 0.37 | 0.37 | 17.5 |
| 10c | 0.33 | 0.42 | 16.4 |

^{a}Cells were incubated with 0.001 to 0.5 mM of xylosides for 96 h and then assayed for cell number. Each dose was tested 5 times.

1-41. (canceled)

42. A pharmaceutical composition comprising:
(a)
at least one compound having the general formula I: 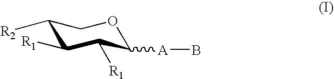
wherein
R₁ groups are same or different and independently selected from N—Y₁Y₂ or O—Y;
Y, Y₁ and Y₂ are independently selected from H, alkyl, C(O)aryl, CH₂aryl, C(O)alkyl, C(O)Oalkyl, C(O)Oalkenyl, where the alkyl and alkenyl groups have 1-100 carbon atoms and the aryl group has 6-100 carbon atoms;
the C(O)Oalkyl and C(O)Oalkenyl includes preferentially all acyls from acetyl (2 carbon atoms) to eicosatetranoyl (20 carbon atoms) with or without single or multiple double bonds in all positions;
R₂ is selected from the group consisting of
R₁, 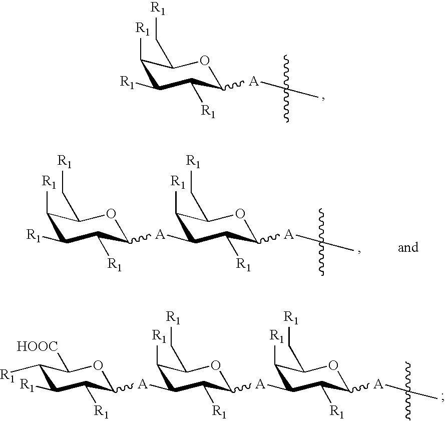
A is O, S, NH or CH₂;
B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y;
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms;
and pharmaceutically acceptable salts thereof, in combination with
(b) non-xylose compounds chosen from
at least one polyamine synthesis inhibitor;
and at least one nitric oxide donor or stimulator of nitric oxide synthesis or inducer of nitric oxide release from S-nitrosothiols;
and optionally also at least at least one anti-tumor agent selected from the group consisting growth factor-receptor interaction inhibitor or heparanase inhibitor and/or cholesterol traffic inhibitors and/or from the group of inducer of epoxygenase and/or inhibitor of topoisomerase and/or cyanide-donor and/or selene-containing compounds; said combinations of compound(s) a) and agents b) being selected such that a synergistic cytotoxic proliferative effect is accomplished.

43. The composition according to claim 42, wherein compound a) is chosen from I, 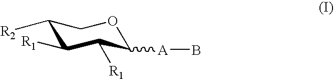
and R₂ is R₁.

44. The composition according to claim 42, wherein compound a) is

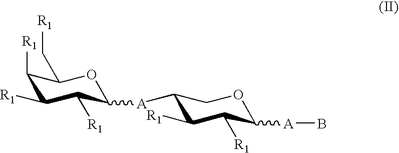

45. The composition according to claim 42, wherein compound a) is

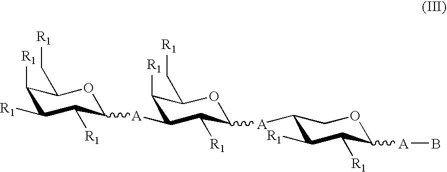

46. The composition according to claim 42, wherein compound a) is

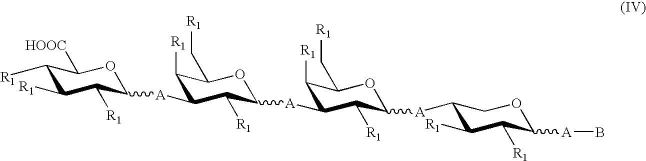

47. The composition according to claim 42, wherein the compounds a) are partially or fully acylated.

48. The composition according to claim 42, wherein alkyl at each occurrence in connection with B has 1-6 carbon atoms.

49. The composition according to claim 42, wherein (a) comprises at least one β-glycoside.

50. The composition according to claim 42, wherein (a) comprises at least one D-xyloside or one D-galactosyl-D-xyloside.

51. The composition according to claim 42, wherein B is naphthyl substituted with at least one OH group.

52. The composition according to claim 51, wherein said substituted naphthyl group is 6-hydroxynaphthyl.

53. The composition according to claim 42, wherein (a) comprises 6-hydroxy-2-naphthyl-β-D-xylopyranoside.

54. The composition according to claim 42, wherein (a) comprises partially or fully acylated 6-hydroxy-2-naphthyl-(β-1,4-D-galactopyranosyl)-β-D-xylopyranoside.

55. The composition according to claim 51, wherein said naphthyl group is substituted with two OH groups.

56. The composition according to claim 55, wherein said substituted naphthyl group is chosen from 5,6-dihydroxynaphthyl, 6,7-dihydroxynaphthyl, 1,4-dihydroxynaphthyl and 5,8-dihydroxynaphthyl.

57. The composition according to claim 56, wherein (a) comprises a β-D-xylopyranoside selected from 5,6-dihydroxynaphthyl-β-D-xylopyranoside, 6,7-dihydroxynaphthyl-β-D-xylopyranoside, 1,4-dihydroxynaphthyl-β-D-xylopyranoside and 5,8-dihydroxynaphthyl-β-D-xylopyranoside.

58. The composition according to claim 42, wherein (a) comprises a partially or fully acylated β-D-galactopyranosyl-O-D-xylopyranoside.

59. The composition according to claim 42, wherein said polyamine synthesis inhibitor is α-difluoromethyl-ornithine (DFMO).

60. The composition according to claim 42, wherein said cholesterol traffic inhibitor is 3β-(2-diethylamino-ethoxy)-androstenone.

61. The composition according to claim 42, wherein said growth factor uptake inhibitor and heparanase inhibitor is suramin.

62. The composition according to claim 42, wherein said NO-donor is spermineNONOate.

63. The composition according to claim 42, wherein said NO-donor is selected from nitroglycerin, S-nitrosothiols and isosorbinid.

64. The composition according to claim 42, wherein said stimulator of NO-production and nitrosothiol formation is selected from interferon-γ and lipopolysaccharide.

65. The composition according to claim 42, wherein said NO-releaser is ascorbate (vitamin C).

66. The composition according to claim 42, wherein said epoxygenase inducer is naphthoflavone.

67. The composition according to claim 42, wherein said topoisomerase inhibitor is etoposide.

68. The composition according to claim 42 wherein (a) is a 6-hydroxy-2-naphthyl substituted glycoside and (b) is DFMO.

69. A composition according to claim 42 wherein (a) is a 6-hydroxy-2-naphthyl substituted glycoside and (b) is a combination of DFMO, spermineNONOate and optionally suramin.

70. A composition according to claim 42 wherein (a) is a 6-hydroxy-2-naphthyl substituted glycoside and (b) is a combination of interferon-γ, lipopolysaccharide and ascorbate (vitamin C).

71. A composition according to claim 42 in combination with amygdalin or prunasin or mandelonitrile or selene.

72. The pharmaceutical composition, comprising the composition according to claim 42, and a pharmaceutically acceptable adjuvant, diluent or carrier.

73. The pharmaceutical composition according to claim 72, in which said composition according to claim 42 is present in an amount such that a dose for each compound (a) and agent (b) in the range 0.001-100 mg/kg body weight is obtained upon administration.

74. A method of treating a proliferative disease in a subject comprising administering to a subject in need thereof an effective amount of the composition according to claim 42.

75. The method according to claim 74, wherein said effective amount is 0.001-100 mg/kg body weight for each compound (a) and (b).

76. The method according to claim 74, wherein said proliferative disease is a tumor disease.

77. The method according to claim 76, wherein said tumor disease is lung cancer, stomach cancer, colon cancer, liver cancer, bladder cancer, prostate cancer, breast cancer or a brain tumor.

78. A compound having the general formula I: 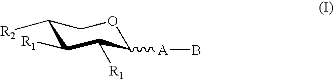
wherein
R₁ groups are the same or different and independently selected from O—Y;
Y is independently selected from C(O)alkyl, C(O)alkenyl, where the alkyl and alkenyl groups have 1-100 carbon atoms;
the C(O)alkyl and C(O)alkenyl includes preferentially all acyls from acetyl (2 carbon atoms) to heneicosanyl (21 carbon atoms) with or without single or multiple double bonds in all positions;
R₂ is selected from the group consisting of R₁, 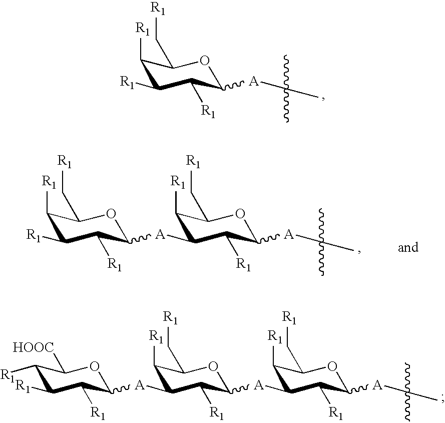
A is O, S, NH or CH₂;
B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y; and
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms.

79. A composition comprising a compound for having the general formula I: 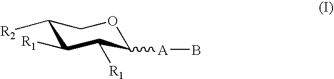
wherein
R₁ groups are the same or different and independently selected from O—Y;
Y is independently selected from C(O)alkyl, C(O)alkenyl, where the alkyl and alkenyl groups have 1-100 carbon atoms;
where the C(O)alkyl and C(O)alkenyl includes preferentially all acyls from acetyl (2 carbon atoms) to heneicosanyl (21 carbon atoms) with or without single or multiple double bonds in all positions;
R₂ is selected from the group consisting of R, 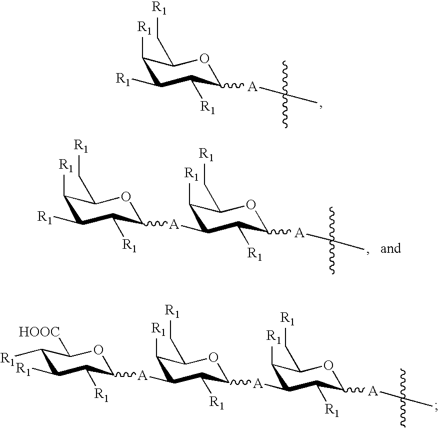
A is O, S, NH or CH₂;
B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y; and
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms.

80. Method of treating a subject having a proliferative disease, comprising administering to a subject in need thereof an effective amount of a compound for having the general formula I: 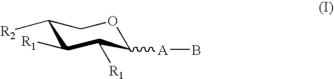
wherein
R₁ groups are the same or different and independently selected from O—Y;
Y is independently selected from C(O)alkyl, C(O)alkenyl, where the alkyl and alkenyl groups have 1-100 carbon atoms;
where the C(O)alkyl and C(O)alkenyl includes preferentially all acyls from acetyl (2 carbon atoms) to heneicosanyl (21 carbon atoms) with or without single or multiple double bonds in all positions;
R₂ is selected from the group consisting of R₁, 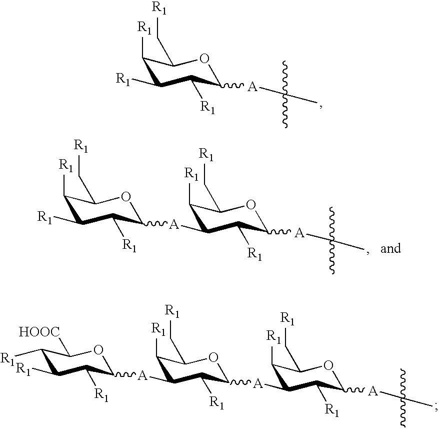
A is O, S, NH or CH₂;
B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y; and
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms.

81. A compound having the general formula I: 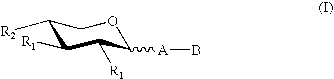
wherein
R₁ groups are same or different and independently selected from N—Y₁Y₂ or O—Y;
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms;
R₂ is selected from the group consisting of R₁, 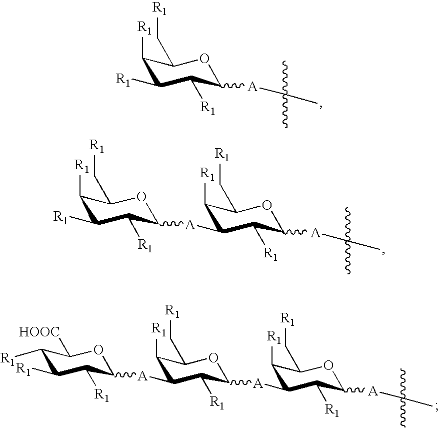
A is O, S, NH or CH₂;
B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y; and
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms; and
with the exclusion of compounds with the general formula I and R₁ is OH, A is O and B is naphthyl with 0, 1 or 2 OH-groups; or R₁ is OH, A is S and B is naphthyl.

82. A composition comprising a compound for having the general formula I: 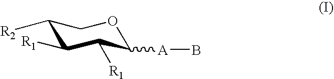
wherein
R₁ groups are same or different and independently selected from N—Y₁Y₂ or O—Y;
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms;
R₂ is selected from the group consisting of R, 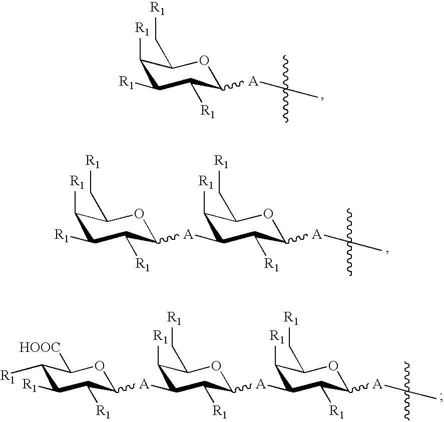
A is O, S, NH or CH₂;
B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y;
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms; and
with the exclusion of compounds with the general formula I and R₁ is OH, A is O and B is naphthyl with 0, 1 or 2 OH-groups; or R₁ is OH, A is S and B is naphthyl.

83. A method of treating a subject having a proliferative disease comprising administering to a subject in need thereof an effective amount of a compound for having the general formula I: 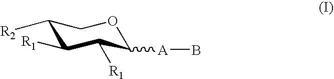
R₁ groups are same or different and independently selected from N—Y₁Y₂ or O—Y;
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms;
R₂ is selected from the group consisting of R₁, 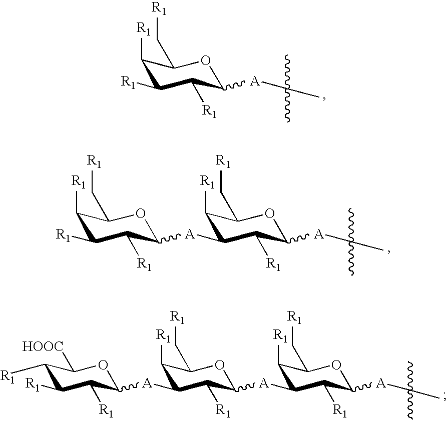
A is O, S, NH or CH₂;
B is selected from naphthyl, naphthylalkyl, anthracenyl, antracenylalkyl or biphenyl, substituted with one or more substituents that are independently selected from F, Cl, Br, I, NO₂, CF₃, COOH, N—Y₁Y₂ or O—Y;
Y, Y₁ and Y₂ are independently selected from H, alkyl, C(O)aryl, CH₂aryl, C(O)alkyl, C(O)Oalkyl, where the alkyl group has 1-100 carbon atoms and the aryl group has 6-100 carbon atoms; and
with the exclusion of compounds with the general formula I and R₁ is OH, A is O and B is naphthyl with 0, 1 or 2 OH-groups; or R₁ is OH, A is S and B is naphthyl.
